(19) United States
(12) Patent Application Publication
(10) Pub. No.: US 2026/0241105 A1
(43) Pub. Date: Aug. 20, 2026

(54) DEVICES, SYSTEMS, AND METHODS FOR DELIVERING MULTICOMPONENT HYDROGELS

(71) Applicant: Clearstream Technologies Limited, Enniscorthy, County Wexford (IE)

(72) Inventors: Matthew CULLEN, Wicklow (IE); Tyler LENT, Mesa, AZ (US); William E. PARMENTIER, Gilbert, AZ (US)

(73) Assignee: Clearstream Technologies Limited, Enniscorthy, County Wexford (IE)

(21) Appl. No.: 19/162,024

(22) PCT Filed: Mar. 8, 2024
(86) PCT No.: PCT/US2024/019092

§ 371 Date: Sep. 3, 2025

Related U.S. Application Data
(60) Provisional application No. 63/489,549, filed on Mar. 10, 2023.

(51) Int. Cl. A61M 5/28; A61M 5/19; A61M 5/31; A61M 5/32

(52) U.S. Cl. CPC A61M 5/284; A61M 5/19; A61M 5/3134; A61M 5/3293; A61M 2005/3201

(57) ABSTRACT

An injection needle assembly includes a hub having a first input port and a second input port, and an outer hub portion rotatably coupled about an inner hub portion having a cam. An elongate hollow stylet extends distally from the hub and includes a proximal end at the hub and distal end spaced a distance from the proximal end. A cannula receives the elongate hollow stylet such that the elongate hollow stylet and the cannula define an inner lumen and an outer lumen separate from the inner lumen, the cannula further including a proximal end and a distal end spaced a distance from the proximal end. The elongate hollow stylet is coupled to the hub such that the proximal end of the elongate hollow stylet engages with the cam and rotation of the outer hub portion translates the elongate hollow stylet between an extended position and a retracted position.

### BACKGROUND

### Field

The present disclosure generally relates to hydrogel delivery systems, and more specifically, to devices, systems, and methods for use associated with a procedure to aid in delivering a multi-component hydrogel.

### Technical Background

Hydrogels are used for a wide variety of applications, such, as for example, radiation spacers. Hydrogels are generally composed of a plurality of components that are mixed prior to application. For example, a polyethyleneimine (PEI) based hydrogel will rapidly form upon coming into contact with serum albumin. It is therefore advantageous to hydrate these two components separately and have the components come into contact with one another at the point of delivery to avoid or minimize clogging of delivery components.

### SUMMARY

An object of the present disclosure is to provide a fluid delivery mechanism including an injection needle assembly having an elongate hollow stylet that is retractable between an extended position and a retracted position. By translating a blunt distal end of the elongate hollow stylet to extend beyond a distal end of an introducer cannula in which the elongate hollow stylet is disposed, it may be possible to minimize the risk of perforating and/or damaging tissue within a target area.

In one embodiment, an injection needle assembly is disclosed. The injection needle assembly includes a hub having a first input port and a second input port, and an outer hub portion rotatably coupled about an inner hub portion having a cam. An elongate hollow stylet extends distally from the hub and includes a proximal end at the hub and distal end spaced a distance from the proximal end. A cannula receives the elongate hollow stylet such that the elongate hollow stylet and the cannula define an inner lumen and an outer lumen separate from the inner lumen, the cannula further including a proximal end and a distal end spaced a distance from the proximal end. The elongate hollow stylet is coupled to the hub such that the proximal end of the elongate hollow stylet engages with the cam and rotation of the outer hub portion translates the elongate hollow stylet between an extended position and a retracted position.

In another embodiment, a fluid delivery system is disclosed. The fluid delivery system includes a dual chamber applicator comprising two chambers separate from one another, each chamber comprising at least one output port on a distal end thereof, and an injection needle assembly. The injection needle assembly includes a hub having a first input port and a second input port, and an outer hub portion rotatably coupled about an inner hub portion having a cam. An elongate hollow stylet extends distally from the hub and includes a proximal end at the hub and distal end spaced a distance from the proximal end. A cannula receives the elongate hollow stylet such that the elongate hollow stylet and the cannula define an inner lumen and an outer lumen separate from the inner lumen, the cannula further including a proximal end and a distal end spaced a distance from the proximal end. The elongate hollow stylet is coupled to the hub such that the proximal end of the elongate hollow stylet engages with the cam and rotation of the outer hub portion translates the elongate hollow stylet between an extended position and a retracted position.

In another embodiment still, a method of translating an elongate hollow stylet in an injection needle assembly is disclosed. The method includes forming a handle on a proximal end of the elongate hollow stylet; disposing the elongate hollow stylet within a cannula, such that a distal end of the elongate hollow stylet extends beyond a distal end of the cannula; engaging the handle with a slot formed in a cam mechanism of an inner hub portion of a hub of the injection needle assembly, the hub further including an outer hub portion rotatably coupled to the inner hub portion; and rotating the outer hub portion relative the inner hub portion, such that the outer hub portion contacts the handle and drives the handle from a first end of the slot to a second end of the slot; wherein translation of the handle from the first end of the slot to the second end of the slot causes the elongate hollow stylet to retract, such that the distal end of the elongate hollow stylet is enclosed within the cannula.

### BRIEF DESCRIPTION OF THE DRAWINGS

The embodiments set forth in the drawings are illustrative and exemplary in nature and not intended to limit the subject matter defined by the claims. The following detailed description of the illustrative embodiments can be understood when read in conjunction with the following drawings, wherein like structure is indicated with like reference numerals and in which:

FIG. 1 depicts a front view of an illustrative delivery system including a material applicator and an injection needle assembly according to one or more aspects shown and described herein;

FIG. 2 depicts a front view of an illustrative delivery apparatus including a material applicator and an injection needle assembly according to one or more aspects shown and described herein;

FIG. 3A depicts a perspective view of an illustrative connector of a material applicator according to one or more aspects shown and described herein;

FIG. 3B depicts a distal view of the illustrative connector of the material applicator of FIG. 3A;

FIG. 4A depicts a perspective view of an illustrative corresponding connector of a dual chamber mixing syringe according to one or more aspects shown and described herein;

FIG. 4B depicts a sectional view of an illustrative hub of a connector, depicting illustrative fluid flow, according to one or more aspects shown and described herein;

FIG. 5A depicts a perspective view of another illustrative corresponding connector of a dual chamber mixing syringe having a side flush port according to one or more aspects shown and described herein;

FIG. 5B depicts a perspective view of an illustrative hub of a connector having a central flush port according to one or more aspects shown and described herein;

FIG. 5C depicts a sectional view of the hub of FIG. 5B;

FIG. 5D depicts a perspective view of another illustrative applicator having a distal end with a side flush port according to one or more aspects shown and described herein;

FIG. 5E depicts a sectional side view of the applicator of FIG. 5D;

FIG. 6A depicts a side view of the connector of FIG. 5A;

FIG. 6B depicts a front view of the connector of FIG. 5A;

FIG. 7 depicts a section view of a delivery apparatus of an injection needle assembly showing an alignment of components according to one or more aspects shown and described herein;

FIG. 8 depicts a side sectional view of an illustrative distal end of an injection needle assembly having a distal mixing chamber and a deployable blunt tip according to one or more aspects shown and described herein

FIG. 9A depicts a perspective view of another illustrative delivery apparatus, according to one more aspects shown and described herein;

FIG. 9B depicts a side view of the delivery apparatus of FIG. 9A, according to one or more embodiments shown and described herein;

FIG. 9C depicts a sectional view along line 9C-9C, of the delivery apparatus of FIGS. 9A and 9B, according to one or more aspects shown and described herein;

FIG. 10A depicts a perspective view of another illustrative delivery apparatus with an elongate hollow stylet in an extended position, according to one more aspects shown and described herein;

FIG. 10B depicts a side view of the delivery apparatus of FIG. 10A, according to one or more embodiments shown and described herein;

FIG. 10C depicts a sectional view along line 10C-10C, of the delivery apparatus of FIGS. 10A and 10B, according to one or more aspects shown and described herein;

FIG. 11A depicts a perspective view of another illustrative delivery apparatus with an elongate hollow stylet in a retracted position, according to one more aspects shown and described herein;

FIG. 11B depicts a side view of the delivery apparatus of FIG. 11A, according to one or more embodiments shown and described herein;

FIG. 11C depicts a sectional view along line 11C-11C, of the delivery apparatus of FIGS. 11A and 11B, according to one or more aspects shown and described herein;

FIG. 12A depicts a schematic of a step of an illustrative process of connecting a hub and extending an elongate hollow stylet of an injection needle assembly according to one or more aspects shown and described herein;

FIG. 12B depicts a schematic of another step of an illustrative process of connecting a hub and extending an elongate hollow stylet of an injection needle assembly, according to one or more aspects shown and described herein; and

FIG. 12C depicts a schematic of another step of an illustrative process of connecting a hub and extending an elongate hollow stylet of an injection needle assembly, according to one or more aspects shown and described herein; and

FIG. 12D depicts a schematic of another step of an illustrative process of connecting a hub and extending an elongate hollow stylet of an injection needle assembly, according to one or more aspects shown and described herein; and

FIG. 12E depicts a schematic of another step of an illustrative process of connecting a hub and extending an elongate hollow stylet of an injection needle assembly, according to one or more aspects shown and described herein; and

FIG. 12F depicts a schematic of another step of an illustrative process of connecting a hub and extending an elongate hollow stylet of an injection needle assembly, according to one or more aspects shown and described herein; and

FIG. 12G depicts a schematic of another step of an illustrative process of connecting a hub and extending an elongate hollow stylet of an injection needle assembly, according to one or more aspects shown and described herein;

FIG. 13A depicts a perspective view of another illustrative delivery apparatus in a blunt tip deployed state, according to one or more aspects shown and described herein;

FIG. 13B depicts a side view of the illustrative delivery apparatus of FIG. 13A in a blunt tip deployed state, according to one or more aspects shown and described herein; and

FIG. 13C depicts a sectional view along line 13C-13C, of the illustrative delivery apparatus of FIGS. 13A and 13B in a blunt tip deployed state, according to one or more aspects shown and described herein.

### DETAILED DESCRIPTION

The present disclosure, in one form, is related to multi-component delivery systems, such as systems for delivering a hydrogel (e.g., a hydrogel spacer) in various treatment procedures. The multi-component delivery systems described herein include a dual lumen syringe that delivers each component along separate paths such that the components mix at the point of delivery. In addition, the delivery systems described herein include auxiliary ports (e.g., a side port or the like) for delivery of flushing fluids or the like and include extendable inner components that include a blunt tip and can be usable to avoid puncturing tissue when the needle is inserted in tissue. The delivery systems described herein include a dual chamber material applicator and an injection needle assembly, which are connectable to each other via one or more quick-connect/disconnect couplings. The delivery systems described herein are configured such that a user can quickly connect a dual syringe applicator to a needle assembly and deliver multi-component materials in a dual syringe system (e.g., components of a hydrogel) such that the components are mixed at the delivery site, the delivery needle can be flushed after delivery of the materials, and/or such that the delivery needle can be blunted after piercing tissue in order to avoid further puncturing tissue at a delivery site (e.g., prostate tissue and/or tissue surrounding a prostate).

As should be appreciated, hydrogels may typically be composed of multiple components that gel (e.g., interact, etc.) upon mixing. Conventional multi-component hydrogels are often mixed prior to application within a target area; however, premature gelation between components may lead to clogging of a delivery device in which the components are disposed, thereby rendering the delivery device ineffective and/or inoperable. Furthermore, these traditional delivery devices often utilize sharp and/or beveled needles for introduction of the hydrogel into the target area, which may pose a risk of inadvertent tissue perforation at the target area.

The disclosed injection needle assembly may improve upon conventional solutions in that the multi-component materials are combined at the point of delivery. That is, the dual lumen syringe utilizes to dispense the various components of the hydrogel may be separately maintained until they are delivered to a target area, such that risk of clogging the injection needle assembly it mitigated. Furthermore, the multi-component materials may be flushed via an auxiliary port incorporated into the hub. In these embodiments, a flushing fluid may be provided to the injection needle assembly via the auxiliary port and flushed through the portions of the dual lumen syringe, thus providing an additional means of avoiding clogging.

It should be further appreciated that the disclosed injection needle assembly may minimize the risk of inadvertent tissue perforation by utilizing an extendable needle that provides a blunted tip upon extension. Accordingly, the needle used to pierce tissue at the target area be selectively blunted to avoid further tissue puncture, as will be described in additional detail herein.

Another advantage of the present disclosure is that the various hubs described herein provide connectors (e.g., quarter turn connectors, threaded nuts, or the like) that are used to couple components together provide a quick and easy way for a user to connect and disconnect components in a manner that ensures that the components are adequately aligned and sealed each time they are connected, even in instances where the needle assembly is inserted within a subject tissue, thereby ensuring the correct mixture and delivery of materials. In addition, an advantage of the various connectors of the present disclosure is that the connectors may provide tactile or audible feedback provided to a user as extra confirmation that the components are correctly aligned and joined when coupled via the connector.

It should be understood that the term “quarter turn” as used herein refers to a rotation that completes about one fourth of a full rotation. That is, a quarter turn rotation rotates about 90° clockwise or counterclockwise. The components described herein are referred to as “quarter turn” components because the components allow about a 90° rotation of components relative to one another.

Embodiments of fluid delivery systems, needle assemblies, and methods using fluid delivery systems will now be described in more detail herein with reference to the drawings, and where like numbers refer to like structures.

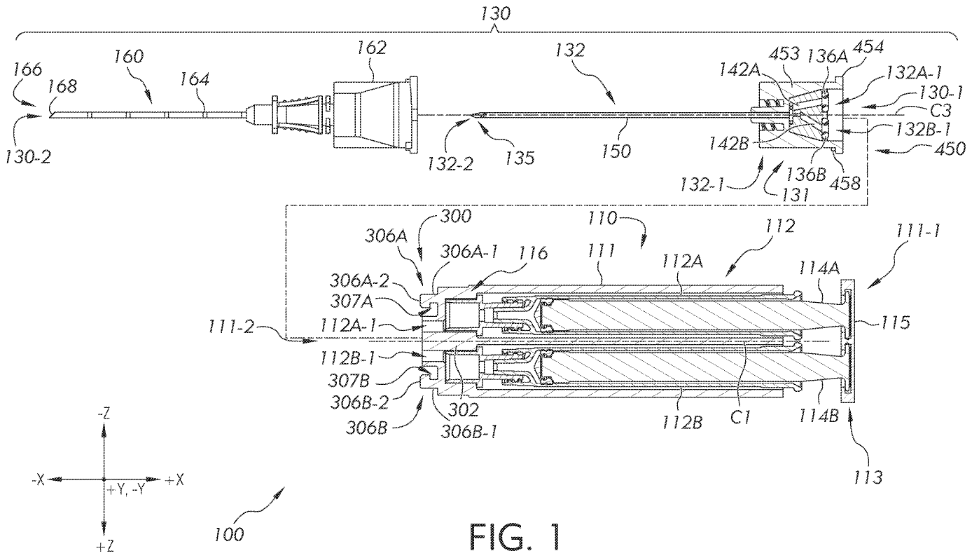

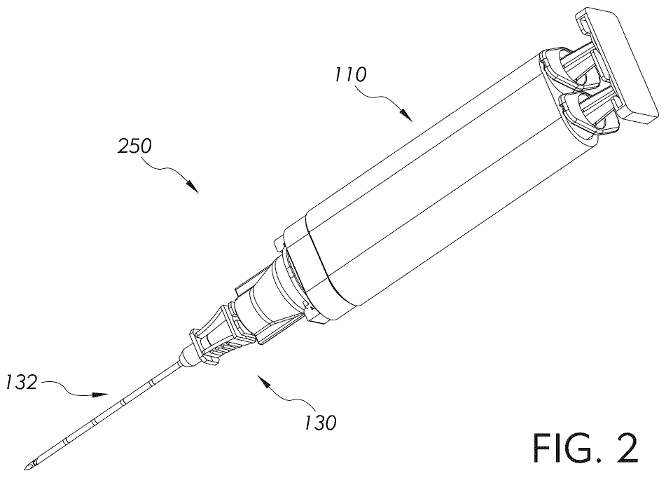

Turning now to the drawings, FIG. 1 depicts an illustrative delivery system 100 according to various embodiments. The delivery system 100, in accordance with an aspect of the present disclosure, may be for use in various procedures where a multi-component material such as a hydrogel is delivered (e.g., as radiation spacers or the like). The delivery system 100 generally includes a material applicator 110 and/or an injection needle assembly 130. The various components for the delivery system 100 are keyed via a connection system such that the material applicator 110 can be quickly and easily coupled to the injection needle assembly 130 for mixing and delivery of components, even in instances where the injection needle assembly 130 is disposed within tissue, as indicated by the dashed lines in FIG. 1 and described herein. When coupled to the injection needle assembly 130, the combination of the injection needle assembly 130 and the material applicator 110 may be referred to herein as a delivery apparatus 250, as depicted in FIG. 2.

Referring to FIG. 2, when the material applicator 110 is coupled to the injection needle assembly 130, the delivery apparatus 250 therefrom has a configuration to facilitate delivery of a flowable multi-component material to an injection site. The multi-component material may be injected while the needle portion of the injection needle assembly 130 is inserted within tissue where delivery of the multi-component material is needed, such as tissue receiving radiation spacers. The injection needle assembly 130 is configured to puncture tissue and deliver the multi-component material at the delivery site.

Referring to FIGS. 1 and 2, optionally, the delivery apparatus 250 may be used in conjunction with an introducer cannula 160. The introducer cannula 160 may facilitate withdrawal of the injection needle assembly 130 of the delivery apparatus 250 from a subject, while the introducer cannula 160 may remain in place to maintain an access path in the tissue to the site, such as for example, to receive and guide a second medical instrument, such as a biopsy device, to the site where the biopsy is to be performed.

Referring again to FIG. 1, the material applicator 110 generally includes a body 111 having a proximal end 111-1 and a distal end 111-2 spaced a distance apart from the proximal end 111-1. The body 111 also defines a pair of syringes 112. The material applicator 110 is configured to separately carry each of a first material component of the multi-component material and a second material component of the multi-component material. The first material component may include, for example, at least two N-hydroxysuccinimide (NHS) ester groups, and the second material component may include, for example, at least two amine groups. For example, the first material component may be a solution containing polyethylene glycol (PEG) succinimidyl succinate and the second material component may be a solution containing albumin and/or polyethylenimine (PEI). In the present embodiment, the first material component and the second material component are combined and mixed within a mixing chamber of the injection needle assembly 130, as will be described in greater detail herein.

The pair of syringes 112 includes an actuator 113, a first component chamber 112A, and a second component chamber 112B. The first component chamber 112A may be, for example, a cylindrical tube that is configured to carry the first material component of the multi-component material. The first component chamber 112A has a first output port 112A-1 (e.g., a first component port). The second component chamber 112B also may be, for example, a cylindrical tube that is configured to carry the second material component of the multi-component material. The second component chamber 112B has a second output port 112B-1 (e.g., a second component port). In some aspects, the first component chamber 112A and the second component chamber 112B are arranged in a substantially longitudinally parallel arrangement.

In some aspects, the actuator 113 includes a first piston 114A, a second piston 114B, and a handle 115. The handle 115 is in the form of a link member that perpendicularly extends between, and is connected to, each of the first piston 114A and the second piston 114B to facilitate simultaneous movement of the first piston 114A and the second piston 114B with the depression or retraction of the handle 115. The first piston 114A is in the form of a plunger that is positioned in the first component chamber 112A proximal to the first material component, and the second piston 114B is in the form of a plunger that is positioned in the second component chamber 112B proximal to the second material component.

The first output port 112A-1 of the first component chamber 112A and the second output port 112B-1 of the second component chamber 112B may be arranged within the distal end 111-2 of the body 111 of the material applicator 110. The first output port 112A-1 and the second output port 112B-1 are generally fluid outputs that are aligned with other ports of other components as described herein such that the first and second material components can be dispensed from and/or received within the respective component chambers 112A, 112B. In some aspects, the first output port 112A-1 may be concentrically aligned with the first component chamber 112A and the second output port 112B-1 may be concentrically aligned with the second component chamber 112B. However, in other aspects, such as the aspect depicted in FIG. 1, the first output port 112A-1 may be located radially inward of a central area of the first component chamber 112A and the second output port 112B-1 may be located radially inward of a central area of the second component chamber 112B such that the first output port 112A-1 and the second output port 112B-1 are as close as possible to a center axis C1 of the body 111 of the material applicator 110 to facilitate alignment with the other components of the delivery system 100 described herein.

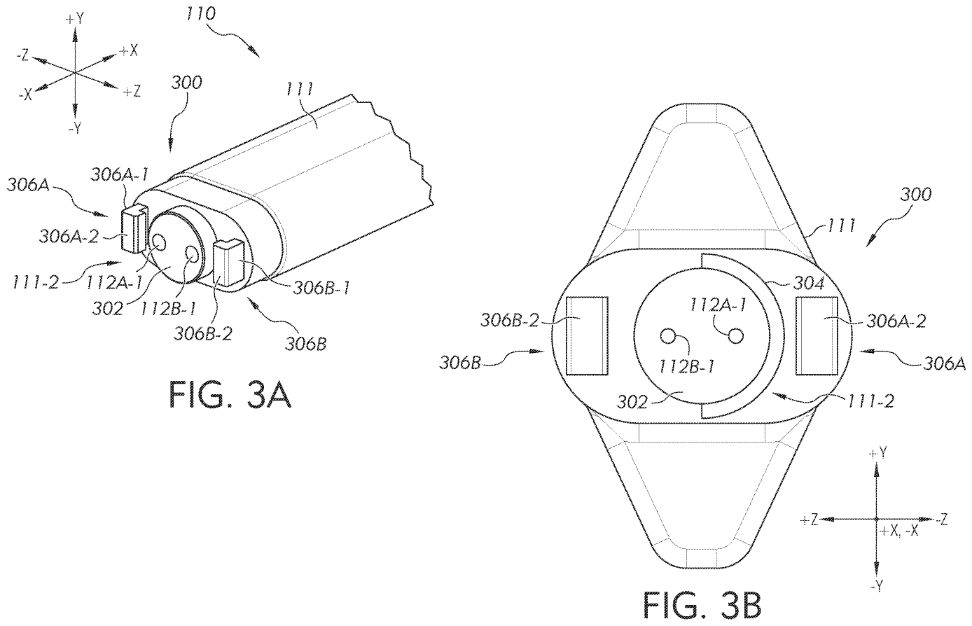

Turning to FIGS. 1, 3A, and 3B, the body 111 of the material applicator 110 further includes a quick connect connector, such as, for example, a quick connect connector 300 integrated with the distal end 111-2 of the material applicator 110. More specifically, as depicted in FIG. 1, the various components of the quick connect connector 300 are integrated with the body 111 such that the quick connect connector 300 and the body 111 are a single monolithic piece. However, it should be understood that this is merely illustrative and the various components of the quick connect connector 300 may be separate pieces that are permanently or semi-permanently joined with the body 111 of the material applicator 110 (e.g., permanently or semi-permanently joined with a distal coupling piece 116 of the material applicator 110).

The quick connect connector 300 is generally located at the distal end 111-2 of the body 111 of the material applicator such that various components of the quick connect connector 300 are positioned adjacent to first output port 112A-1 and the second output port 112B-1. As will be described herein, the quick connect connector 300 is generally shaped and sized to releasably interlock with a corresponding quarter turn connector 400 of the material applicator 110 and/or with a connector 450 of the injection needle assembly 130. As will be described in greater detail herein, when the injection needle assembly 130 or the material applicator 110 is coupled to the material applicator 110 via the quarter turn connectors 300, 400, 450 thereof, the various ports thereof are aligned and sealed with the first output port 112A-1 and the second output port 112B-1 of the material applicator 110.

Still referring to FIGS. 1, 3A, and 3B, the quick connect connector 300 of the material applicator includes a circular protrusion 302 extending distally (e.g., in the −x direction of the coordinate axes of FIG. 3A) from the distal end 111-2 of the material applicator 110, a semi-circular channel 304 (FIG. 3B) disposed within the distal end 111-2 of the material applicator 110 along a periphery of the circular protrusion 302, and a pair of bayonet coupling members (e.g., a first coupling member 306A and a second coupling member 306B) disposed radially outward of the circular protrusion 302 and the semi-circular channel 304. As shown in FIGS. 1, 3A, and 3B, the first output port 112A-1 and the second output port 112B-1 are disposed within the circular protrusion 302. That is, the openings into the first component chamber 112A and the second component chamber 112B are located on the circular protrusion 302.

The circular protrusion 302 is generally shaped and sized to correspond to a recess formed in the injection needle assembly 130 and the material applicator 110, as described in greater detail herein. The circular protrusion may generally be disposed in or around a central area of the distal end 111-2 of the body 111. In some embodiments, the circular protrusion 302 may be concentric with the body such that the center axis C1 of the body 111 extends through a center of the circular protrusion 302. The distance that the circular protrusion 302 extends away from the distal end 111-2 of the body is generally a distance that corresponds to a depth of the recess formed in the injection needle assembly 130 and the material applicator 110 such that the circular protrusion 302 can be completely inserted therein, but is otherwise not limited by the present disclosure.

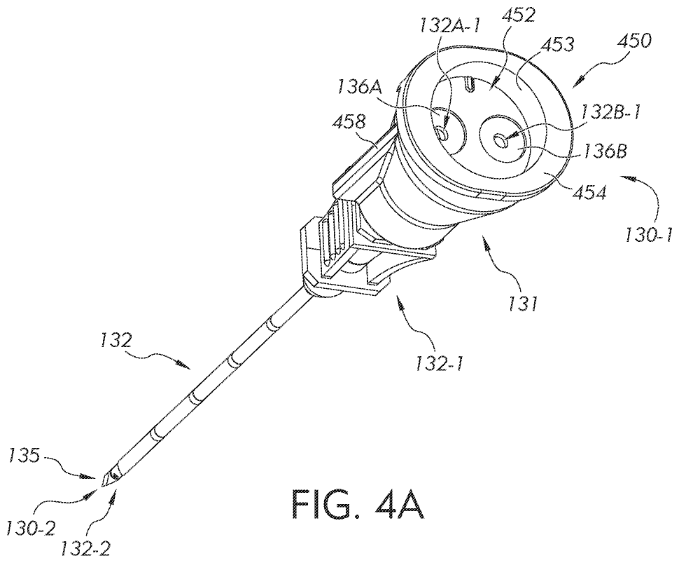

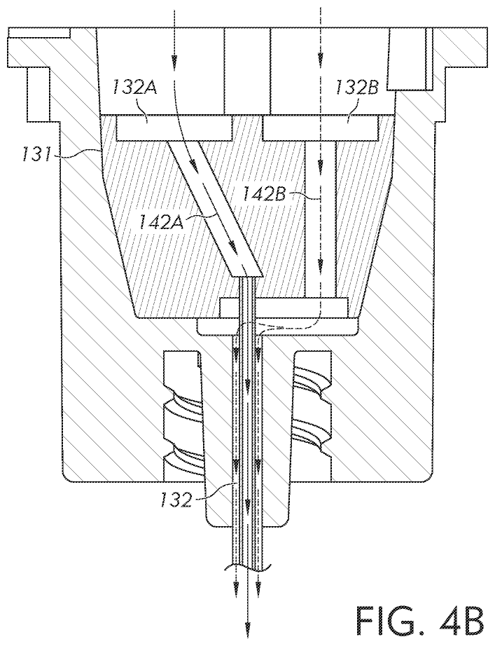

As particularly depicted in FIGS. 3A-3B, the semi-circular channel 304 is disposed around the periphery of the circular protrusion 302 such that the semi-circular channel 304 defines an outer edge of half of the circular protrusion 302. That is, the semi-circular channel 304 extends from a first side (e.g., in the ty direction of the coordinate axes of FIGS. 3A-3B) around the perimeter of the circular protrusion 302 to a second side (e.g., in the −y direction of the coordinate axes of FIGS. 3A-3B). The semi-circular channel 304 is generally shaped and sized to receive a quarter circular extension 456 (FIG. 4A) of the corresponding quarter turn connector 400 of the material applicator 110 (FIG. 1) or the quarter circular extension 456 of the connector 450 of the injection needle assembly 130, as described in greater detail herein. While the aspects depicted in FIGS. 3A-3B depict the semi-circular channel 304 as extending a full semi-circle, the present disclosure is not limited to such. That is, the semi-circular channel 304 may completely surround the circular protrusion 302 in other aspects (e.g., a full circular channel) or may only surround a portion of the circular protrusion 302 to an extent that is lesser than or greater than the semi-circular aspect depicted in FIGS. 3A-3B (e.g., a quarter circular channel or the like). However, it should be understood that, to ensure appropriate keying such that the various components of the delivery system 100 (FIG. 1) are appropriately aligned and rotatable via the respective quarter turn connectors 300, 400, 450, the channel corresponds in size to the size of the extensions.

Referring again to FIGS. 1, 3A, and 3B, the first coupling member 306A and the second coupling member 306B each extend from the distal end 111-2 of the body 111 of the material applicator 110 and are generally shaped and sized to retain the material applicator 110 or the injection needle assembly 130 when coupled to the material applicator 110. Each of the first coupling member 306A and the second coupling member 306B may be a bayonet style coupling member, an L-beam coupling member, or the like. For example, as particularly depicted in FIG. 1, the first coupling member 306A extends distally at a particular distance from the distal end 111-2 of the body 111, turns about 90 degrees, and extends inward toward the center axis C1 of the body 111, resulting in a first extension piece 306A-1 that extends in a direction generally coplanar with the center axis C1 of the body 111 (e.g., along the x-axis of the coordinate axes of FIGS. 1, 3A, and 3B) and a second extension piece 306A-2 that extends in a direction that is generally perpendicular to the center axis C1 of the body (e.g., along the z-axis of the coordinate axes of FIGS. 1, 3A, and 3B), thereby defining a pocket 307A between the distal end 111-2 of the body 111 and the second extension piece 306A-2. Similarly, the second coupling member 306B extends distally at a particular distance from the distal end 111-2 of the body 111, turns about 90 degrees, and extends inward toward the center axis C1 of the body 111, resulting in a first extension piece 306B-1 that extends in a direction generally coplanar with the center axis C1 of the body 111 (e.g., along the x-axis of the coordinate axes of FIGS. 1, 3A, and 3B) and a second extension piece 306B-2 that extends in a direction that is generally perpendicular to the center axis C1 of the body 111 (e.g., along the z-axis of the coordinate axes of FIGS. 1, 3A, and 3B), thereby defining a pocket 307B between the distal end 111-2 of the body 111 and the second extension piece 306B-2.

As depicted in FIGS. 1, 3A, and 3B, the first coupling member 306A and the second coupling member 306B are located opposite one another, radially outward of the circular protrusion 302 and the semi-circular channel 304. However, this is merely illustrative, and other locations and spacing are contemplated and included within the scope of the present disclosure. Further, while the present aspect includes a pair of coupling members (e.g., the first coupling member 306A and the second coupling member 306B), this is also merely illustrative. That is, other amounts of coupling members are also contemplated and included within the scope of the present disclosure.

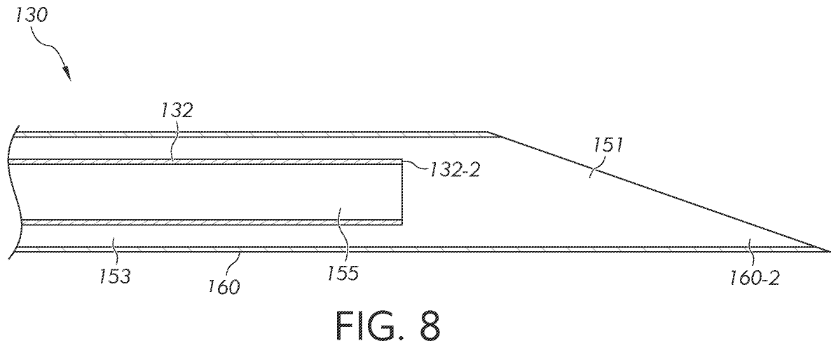

It should be appreciated that other quick connect/disconnect devices and components may be used without departing from the scope of the present disclosure. For example, a threaded nut type connector may be used, as depicted, for example, in FIGS. 8A-11C.

Referring now to FIGS. 1 and 4A, the injection needle assembly 130 has a proximal end 130-1 that extends proximally (in the +x direction of the coordinate axes of FIG. 1) and a distal end 130-2 that extends distally (e.g., in the −x direction of the coordinate axes of FIG. 1). The injection needle assembly 130 generally includes a hub 131 and an elongate hollow stylet 132 that extends distally (e.g., in the −x direction of the coordinate axes of FIG. 1) from hub 131. The elongate hollow stylet 132 has a proximal end 132-1 and a distal end 132-2. The hub 131 is fixedly attached, e.g., through overmolding, adhesive and/or pressed fit, to the proximal end 132-1 of the elongate hollow stylet 132. The hub 131 includes a plurality of input ports (e.g., a first input port 132A-1 and a second input port 132B-1) of the injection needle assembly 130. The hub 131 is configured for removable connection to the material applicator 110 via the connector 450 such that, when connected, the first input port 132A-1 of the injection needle assembly 130 is aligned and sealed with the first output port 112A-1 of the material applicator 110 and the second input port 132B-1 of the injection needle assembly 130 is aligned and sealed with the second output port 112B-1 of the material applicator 110, as described in greater detail herein.

The elongate hollow stylet 132 of injection needle assembly 130 is configured to facilitate fluid communication with the plurality of output ports 112A-1, 112B-1 of the material applicator 110 so as to receive the two components of the multi-component material from material applicator 110 and direct the two components to the distal end 132-2 thereof for mixing and delivery. In some embodiments, the distal end 132-2 has a closed distal end and a plurality of side ports (e.g., two, three, or more) proximal to the closed distal end. The closed distal end of the elongate hollow stylet 132 may be, for example, a closed stylet needle tip.

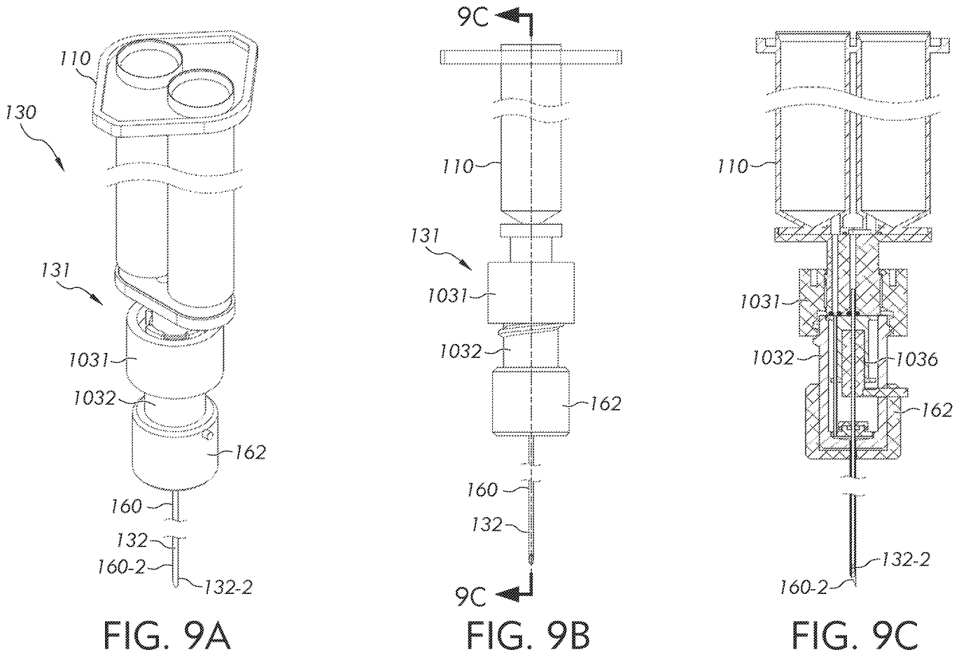

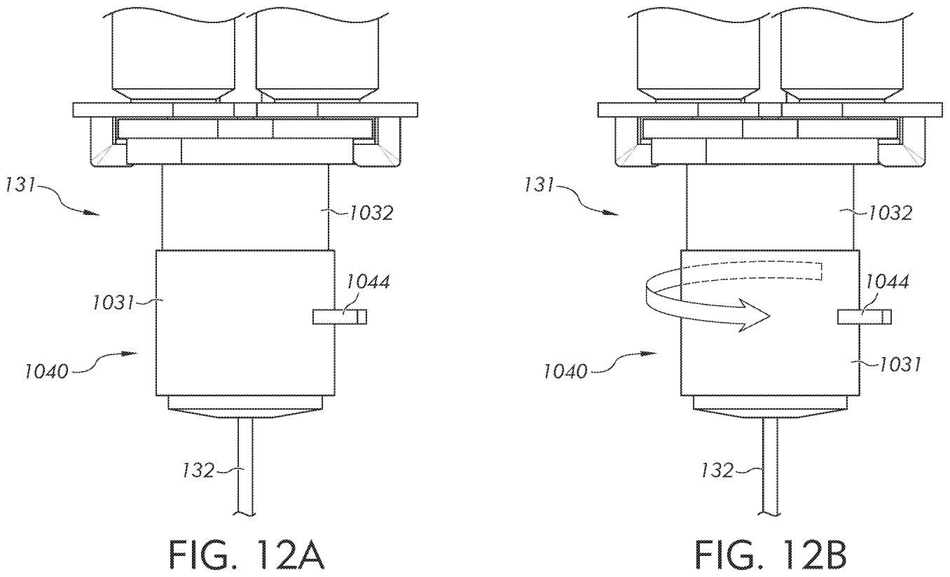

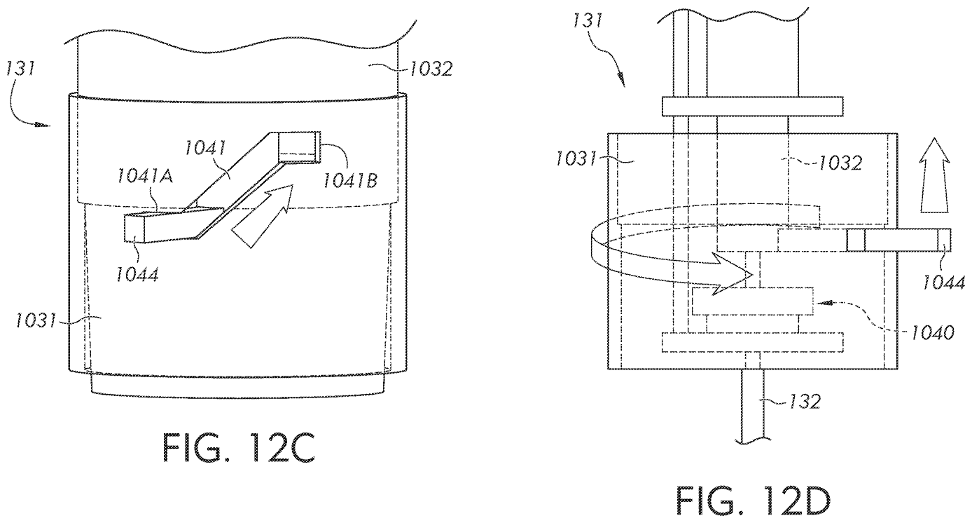

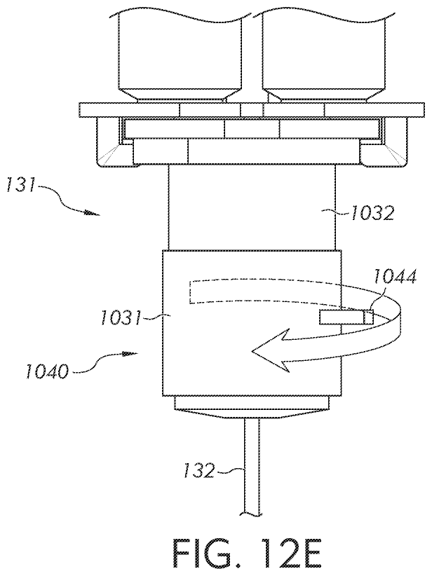

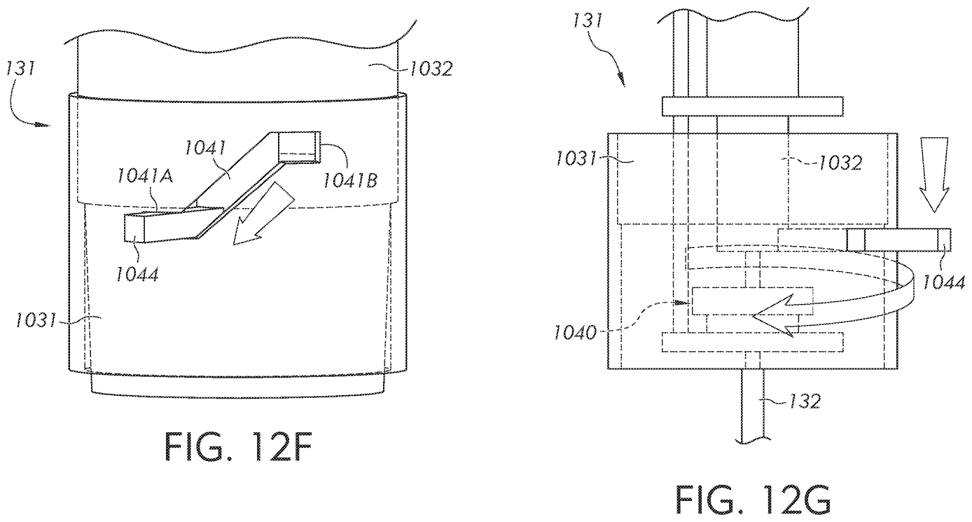

Referring again to FIG. 1, the elongate hollow stylet 132 may be constructed, for example, by an elongate cannula 150 being fixedly connected to the closed stylet needle tip, with the plurality of side ports being located in the elongate cannula 150 in a distal chamber thereof that is immediately proximal to the closed stylet needle tip. More particularly, the elongate cannula 150 of the elongate hollow stylet 132 defines an outer side wall that surrounds an outer lumen of the elongate hollow stylet 132. In addition, located within the outer lumen of the elongate cannula 150 is an inner side wall that surrounds an inner lumen, the inner lumen being concentric with the outer lumen such that a passageway is defined between the inner side wall and the outer side wall. Furthermore, a distal opening of the outer lumen and a distal opening of the inner lumen are open to the distal chamber of the elongate hollow stylet 132. As depicted in FIG. 12, for example, the outer side wall may end in a piercing tip that is usable to pierce tissue and the inner side wall may end in a blunt tip. The inner side wall is extendable so that the blunt tip extends distally beyond the piercing tip, thereby effectively blunting the distal end of the elongate hollow stylet 132 so that the piercing tip cannot pierce tissue when the blunt tip is extended. Mechanisms for extending the blunt tip are depicted, for example, in FIGS. 9-14C.

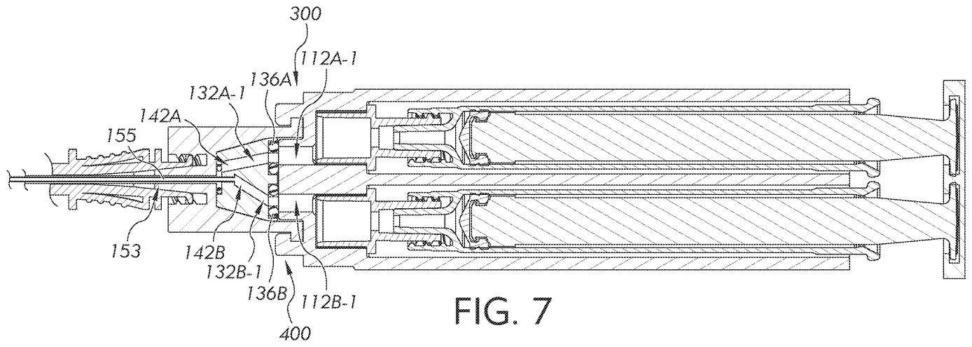

Referring to FIGS. 1 and 4A-4B, 6A-6B, and 7, the hub 131 may further include a plurality of passageways therein for fluidly coupling the first input port 132A-1 and the second input port 132B-1 to the distal chamber 151. For example, in some aspects, a first channel 142A may couple the first input port 132A-1 to the outer lumen 153 by extending through the hub 131 from the first input port 132A-1 to the outer lumen 153. In another example, a second channel 142B may couple the second input port 132B-1 to the inner lumen 155 by extending through the hub 131 from the second input port 132B-1 to the inner lumen 155. The positioning of the first channel 142A and the second channel 142B may be dependent on a location of the first input port 132A-1 and the second input port 132B-1. For example, the first input port 132A-1 and the second input port 132B-1 are spaced to be aligned with the output ports 112A-1, 112B-1 of the material applicator 110. As such, the first input port 132A-1 and the second input port 132B-1 are generally spaced the same distance radially outward from the center axis C3 of the injection needle assembly 130. In such an aspect, the first channel 142A may extend longitudinally toward the distal end 132-2 of the injection needle assembly 130 (e.g., parallel with the center axis C3 of the needle assembly) and couple to the outer lumen near a distal end of the hub 131. The second channel 142B may extend at an angle with respect to the center axis C3 from the second input port 132B-1 to the inner lumen 155. However, it should be understood that the present disclosure is not limited to such. For example, in another aspect, an alternative first channel may have a first channel portion that extends longitudinally through the alternative hub and a second channel portion that extends laterally (e.g., transverse to the first channel portion) to fluidly couple the first input port to the outer lumen. An alternative second input port may be located at the center of the alternative hub and an alternative second channel may extend longitudinally from the alternative second input port to the inner lumen.

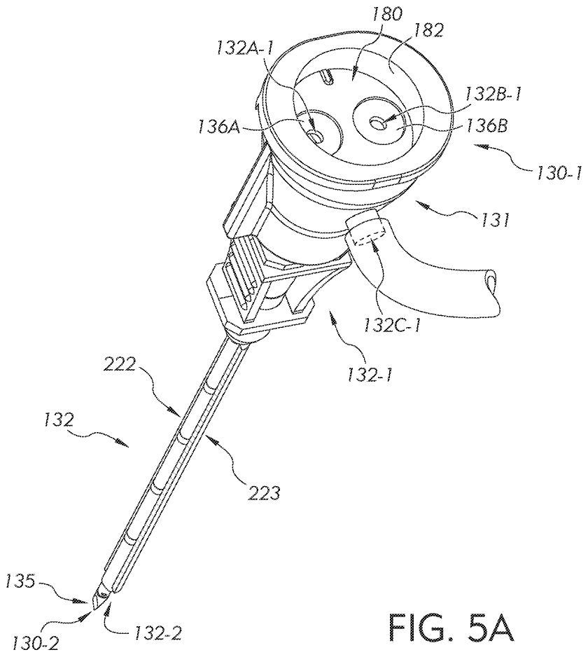

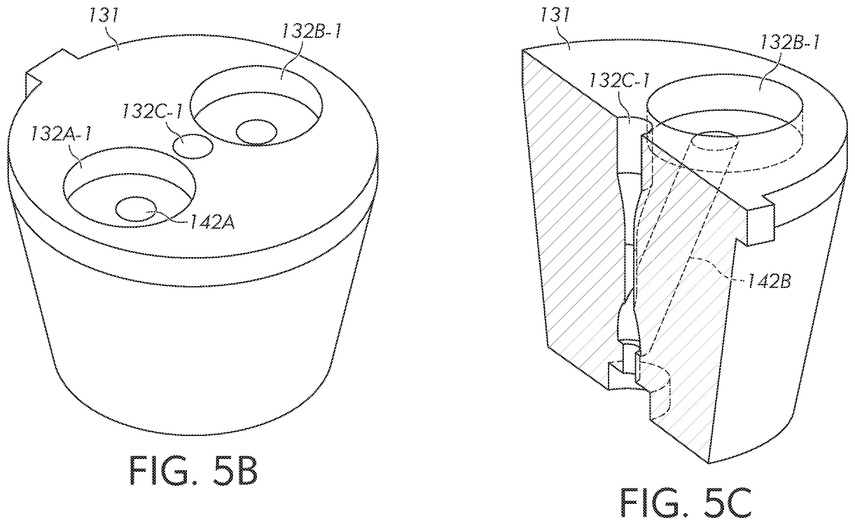

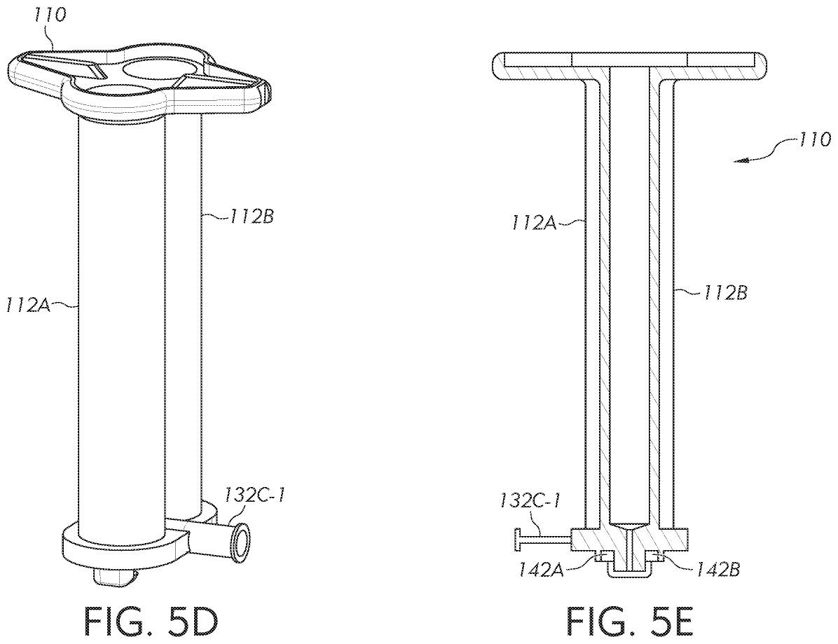

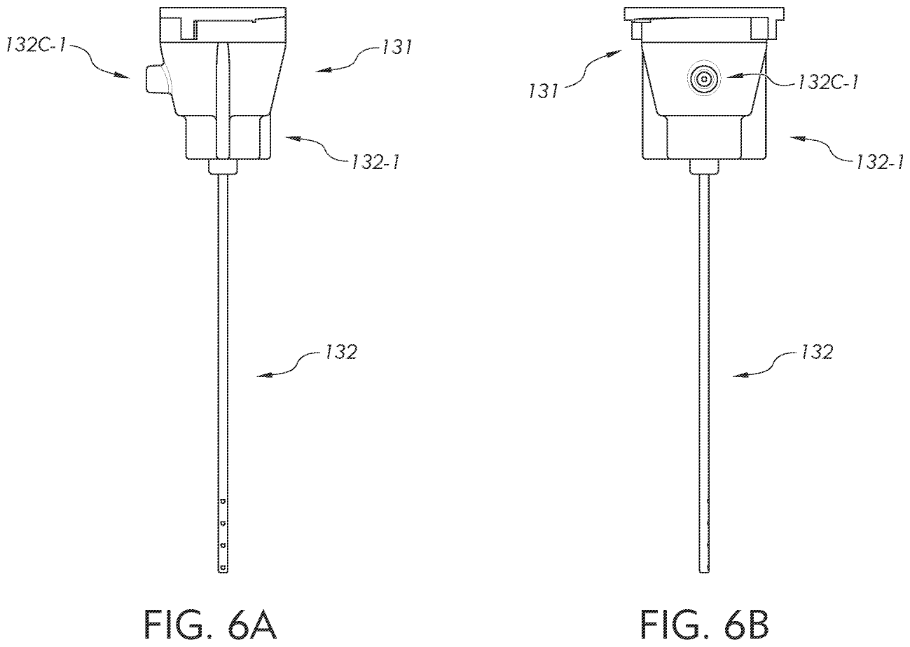

In an embodiment, these features of the injection needle assembly 130 (e.g., the elongate hollow stylet 132 including the inner lumen 155, the outer lumen 153, the distal chamber 151, and the plurality of side ports may advantageously result in an assembly that separately delivers the separate components of the multi-component material to the point where the material is to be applied such that the components are not mixed together until they reach the point where the material is to be applied, thereby avoiding issues relating to clogging or the like. However, should clogging occur, a flushing medium may be dispensed to the various lumens via an auxiliary port, such as the auxiliary ports depicted in FIGS. 5A-5E and 6A-6B.

The injection needle assembly 130 may be used in conjunction with the introducer cannula 160 (also sometimes referred to in the art as a coaxial introducer needle) to allow elongate hollow stylet 132 to be removed from the introducer cannula 160, while maintaining access to the procedure site with the coaxial introducer needle. That is, the delivery apparatus 250 may be removed from the introducer cannula 160 and replaced with a variety of other instruments, such as a biopsy device or another stylet. In some aspects, the introducer cannula 160 has a coaxial hub 162, a coaxial cannula 164, a cannula lumen 166 and a distal annular rim 168. The cannula lumen 166, for example, is configured (e.g., having a cylindrical shape) to receive the elongate hollow stylet 132 of the injection needle assembly 130. When the elongate hollow stylet 132 of the injection needle assembly 130 is fully inserted into the cannula lumen 166 of the introducer cannula 160 (e.g., distal movement of the elongate hollow stylet 132 is stopped by contact of the hub 131 of the injection needle assembly 130 with the coaxial hub 162 of the introducer cannula 160, the plurality of side ports 134 of the elongate hollow stylet 132 are located distal to the distal annular rim 168 of the introducer cannula 160. That is, the distal end 132-2 of the elongate hollow stylet 132 extends beyond the distal annular rim 168 such that the introducer cannula 160 does not block the plurality of side ports 134.

Furthermore, although not depicted, it should be further appreciated that, in some embodiments, the injection needle assembly 130 may be further used without introducer cannula 160. For example, in these embodiments, the distal end 132-2 of the elongate hollow stylet 132 may include a beveled and/or sharpened distal tip configured to provide access to a target area.

Referring to FIGS. 1 and 4A, the connector 450 of the injection needle assembly 130 includes a circular recess 452 sized to receive the circular protrusion 302 on the distal end 111-2 of the material applicator 110 and a ramped lip 454 extending radially outward of the circular recess 452. The circular recess 452 is generally a recess that is defined by a wall 453 extending proximally (e.g., in the +x direction of the coordinate axes of FIG. 1) from a proximal end 130-1 of the injection needle assembly 130 (e.g., extending proximally from the hub 131 of the injection needle assembly 130). The wall 453 extends around the center axis C3 of the injection needle assembly 130 to form the circular recess 452. The wall 453 may be shaped and sized such that the circular recess 452 formed thereby corresponds to the shape and size of the circular protrusion 302 of the material applicator 110. In some aspects, the wall 453 may be an extension of sidewalls of the hub 131 of the injection needle assembly 130.

The plurality of input ports 132A-1, 132B-1 extend out of the proximal end 130-1 of the injection needle assembly 130 within the circular recess 452. That is, the circular recess 452 includes the plurality of input ports 132A-1, 132B-1 therein. In some aspects, the circular recess 452 may include one or more features (e.g., additional recesses, retention pieces, channels, etc.) that are adapted to hold at least one seal around the plurality of input ports 132A-1, 132B-1. For example, a first seal 136A may be held within the circular recess 452 around the first input port 132A-1 and a second seal 136B may be held within the circular recess 452 around the second input port 132B-1. The seals 136A, 136B may each be any seal that allows the injection needle assembly 130 to form a seal with the material applicator 110 when brought together as described herein such that the first input port 132A-1 is joined and sealed with the first output port 112A-1 (e.g., to form a fluid coupling between the first input port 132A-1 and the first output port 112A-1) and the second input port 132B-1 is joined and sealed with the second output port 112B-1 (e.g., to form a fluid coupling between the second input port 132B-1 and the second output port 112B-1). For example, the seals may be O-rings, stadium shaped seals, oval seals, and/or the like. While a single seal is depicted herein for each port, the present disclosure is not limited to such. For example, a single seal, such as a figure eight shaped gasket or the like, may be used to individually seal the ports as described herein. However, it should be understood that the ports (e.g., the first input port 132A-1 and the second input port 132B-1) remain sealed from one another to avoid premature combining of components prior to reaching the distal chamber 151 (FIG. 4D).

Still referring to FIGS. 1 and 4A, the ramped lip 454 is generally formed by the wall 453 that defines the circular recess 452. That is, the ramped lip 454 generally extends radially outward from the wall 453. The ramped lip 454 is shaped and sized to be received and retained by the pair of coupling members 306A, 306B when the connector 450 is rotated relative to the quick connect connector 300, as described herein. That is, the ramped lip 454 has a first thickness (e.g., as defined along the x-axis of the coordinate axes of FIG. 1) at one or more first portions thereof, which gradually increases to a second thickness (e.g., as defined along the x-axis of the coordinate axes of FIG. 1) at one or more second portions thereof to allow for compressed holding of the ramped lip 454 by the pair of coupling members 306A, 306B within the pockets 307A, 307B thereof. In some aspects, the ramped lip 454 may extend around an entire periphery of the wall 453. In other aspects, the ramped lip may extend only around a portion of the periphery of the wall 453. In such aspects, a plurality of ramped lips may be used.

In some aspects, the connector 450 further includes a pair of stops 458 (only one depicted in FIG. 4A). Each one of the pair of stops 458 extends distally (e.g., in the −x direction of the coordinate axes of FIG. 4A, toward the distal end 132-2 of the injection needle assembly 130) from the ramped lip 454. Each one of the pair of stops 458 is aligned on the connector 450 such that the pair of stops 456 contact the coupling members 306A, 306B during a quarter turn rotating motion to hinder further rotational movement of the connector 450 relative to the quick connect connector 300 beyond a quarter rotation. It should be understood that any number of stops may be used, though the number of stops generally corresponds to the same number of coupling members (or less than the number of coupling members). For example, if the quick connect connector 300 includes two coupling members 306A, 306B, the connector 450 may include one stop 456 or two stops 456. In some aspects, the pair of stops 456 may extend radially outward from the wall 453 and may not be connected to the ramped lip 454.

In an embodiment, the features of the various connectors may advantageously result in a structure that allows a user to quickly connect and subsequently disconnect components in such a manner that a user can easily confirm that components are correctly sealed and aligned with one another to ensure the correct materials held within are mixed together and then subsequently delivered to a site on a subject. This improvement is shown in the operation of the various components of the delivery system 100 (FIG. 1). Furthermore, it should be understood that the various connectors described herein are merely illustrative and other connectors are also contemplated and included within the scope of the present disclosure.

As noted herein, in some embodiments, the hub 131 may further include an auxiliary port, which may be configured to provide a flushing fluid, or other similar fluid, to the injection needle assembly 130. Illustrative examples of auxiliary ports are depicted in FIGS. 5A-5E and 6A-6B. In some embodiments, the auxiliary ports depicted may be coupled to an auxiliary applicator (not shown). In these embodiments, an illustrative auxiliary applicator generally includes a body having a proximal end and a distal end spaced a distance apart from the proximal end. The body also defines a syringe. The syringe is configured to carry a material, such as a hydro-dissection solution (e.g., saline or another hydro-dissection solution), an anesthetic solution, and/or the like. The syringe includes an actuator and a chamber. The chamber may be, for example, a cylindrical tube that is configured to carry the hydro-dissection solution, the anesthetic solution, and/or the like. The chamber has an output port. In some aspects, the actuator includes a piston 124 and a handle. The handle is connected to the piston at a proximal end thereof to facilitate movement of the piston with the depression or retraction of the handle. The piston is in the form of a plunger that is positioned in the chamber proximal to the hydro-dissection solution, the anesthetic solution, and/or the like. The output port of the chamber may be arranged within the distal end of the body of the applicator. The output port is generally a fluid output that is fluidly coupled to an auxiliary port of the hub such that the fluid within the chamber is deliverable via the auxiliary port of the hub. In some aspects, the output port may be couplable to the auxiliary port via tubing or the like.

Furthermore, although not depicted, in some embodiments, the auxiliary applicator may be in the form of a third component chamber may be formed on the syringe 112 and fluidly coupled to the hub 131. Accordingly, in these embodiments, depression of the actuator 113 may cause a flushing fluid (e.g., hydro-dissection solution, etc.) to be dispensed from the third component chamber, through the hub 131, and into the elongate hollow stylet 132.

Turning now to FIGS. 5A-5E and 6A-6B, various embodiments of the auxiliary input port 132C-1 are depicted in additional detail. For example, as illustrated in FIGS. 5A, 6A, and 6B, the auxiliary input port 132C-1 may be located on a side of the body of the hub 131. That is, the auxiliary input port 132C-1 is depicted as being an opening in an outer wall of the hub 131. However, the location of the auxiliary input port 132C-1 is not limited by the present disclosure and may be positioned at any location on the hub 131. In some embodiments, the auxiliary input port 132C-1 may be positioned such that an object fluidly coupled thereto (e.g., connective tubing, the material applicator 110, or the like) can be coupled without hindering the coupling of the material applicator 110 to the hub 131, as described herein. In some embodiments, the auxiliary input port 132C-1 may be located within a protrusion or the like that is shaped, sized, and/or configured to couple to an object such as connective tubing, the material applicator 110, or the like.

For example. As illustrated in FIGS. 5B and 5C, the auxiliary input port 132C-1 may be formed within a top surface of the hub 131, such that the auxiliary input port 132C-1 is positioned between the first input port 132A-1 and the second input port 132B-1. As shown most clearly in FIG. 5C, the auxiliary input port 132C-1 may extend through the hub 131 such that the auxiliary input port 132C-1 may be fluidly coupled to each of the first channel 142A and second channel 142B. Accordingly, when a flushing fluid is provided to the auxiliary input port 132C-1, the flushing fluid may traverse both the first channel 142A and the second channel 142B to aid in removing any blockages and/or clogging within the injection needle assembly 130.

Furthermore, in some embodiments, the auxiliary input port 132C-1 may be formed as a portion of the material applicator 110, as illustrated in FIGS. 5D and 5E. In these embodiments, the auxiliary input port 132C-1 may be fluidly coupled to each of the first component chamber 112A and the second component chamber 112B, such that flushing fluid supplied to the auxiliary input port 132C-1 may pass through the first component chamber 112A and second component chamber 112B and into the first channel 142A and second channel 142B, respectively. It should be appreciated that, in these embodiments, the auxiliary applicator (not depicted) may supply the flushing fluid to the auxiliary input port 132C-1 at a sufficient pressure and/or flow rate to traverse the injection needle assembly 130. However, in the embodiment depicted in FIGS. 5D and 5E, the handle 115 of the material applicator 110 may be further utilized to force the flushing fluid through the first component chamber 112A and second component chamber 112B once the flushing fluid has been supplied to the material applicator 110 via the auxiliary input port 132C-1.

In an embodiment, these features of the injection needle assembly 130 (e.g., the elongate hollow stylet 132 including the various lumens, the distal chamber, and the port(s)) may advantageously result in an assembly that separately delivers the separate components of the multi-component hydrogel to the point where the hydrogel is to be applied such that the components are not mixed together until they reach the point where the spacer is to be located, thereby avoiding issues relating to clogging or the like. Further, the features of the injection needle assembly 130 may advantageously result in an assembly where the same assembly can be used to distribute another fluid, such as saline a hydro-dissection solution, or the like, to a target site before or after distribution of the multi-component hydrogel without the need to insert a different device into the space, disconnect and connect different components, and/or the like.

While the present disclosure generally relates to the elongate cannula 150 having two separate passageways, each of which is fluidly coupled to a respective port (e.g., the first input port 132A-1, the second input port 132B-1, and the auxiliary input port 132C-1 coupled to both, the present disclosure is not limited to such. That is, in some embodiments, the elongate cannula 150 may include three or more concentric lumens whereby one of the lumens is coupled to any number of the ports. In some embodiments, the auxiliary input port 132C-1 may be capped off after delivery of the material so that other materials do not backflow through the auxiliary input port 132C-1. Other configurations should be understood and appreciated.

Referring now to FIG. 8, another embodiment of an injection needle assembly is depicted. It should be appreciated that the injection needle assembly 130 is similar to the injection needle assembly 130 described herein with reference to FIGS. 1-7. As such, like structure is indicated with like reference numerals. Furthermore, in addition to the components described herein, it should be appreciated that the injection needle assembly 130 may further include any and/or all of the components of the injection needle assembly 130 depicted in FIGS. 1-7.

For example, in these embodiments, the injection needle assembly 130 may include an introducer cannula 160 having a proximal end 160-1 and a distal end 160-2, with the distal end 160-2 spaced a distance from the proximal end 160-1. As further depicted in FIG. 8, the distal end 160-2 may be a beveled, or otherwise sharpened, distal end, such that the distal end 160-2 of the introducer cannula 160 may be used to pierce tissue at a target area.

As further depicted in FIG. 8, the injection needle assembly 130 may further include a elongate hollow stylet 132 having a proximal end 132-1 and a distal end 132-2 spaced a distance from the proximal end 132-1. In these embodiments, the distal end 132-2 of the hollow needle may be a blunt, open distal end.

Referring still to FIG. 8, the elongate hollow stylet 132 may be disposed within the introducer cannula 160 such that the introducer cannula 160 and the elongate hollow stylet 132 define two separate lumens. For example, the elongate hollow stylet 132 may define a hollow needle lumen, such as an inner lumen 155 (e.g., extending within and through the elongate hollow stylet) and an introducer cannula lumen (e.g., outer lumen 153) may be defined between the elongate hollow stylet 132 and the introducer cannula 160 when the elongate hollow stylet 132 is disposed within the introducer cannula 160. In the embodiments described herein, the introducer cannula lumen 153 and the hollow needle lumen 155 may generally correspond to the inner lumen and outer lumen depicted in FIG. 8.

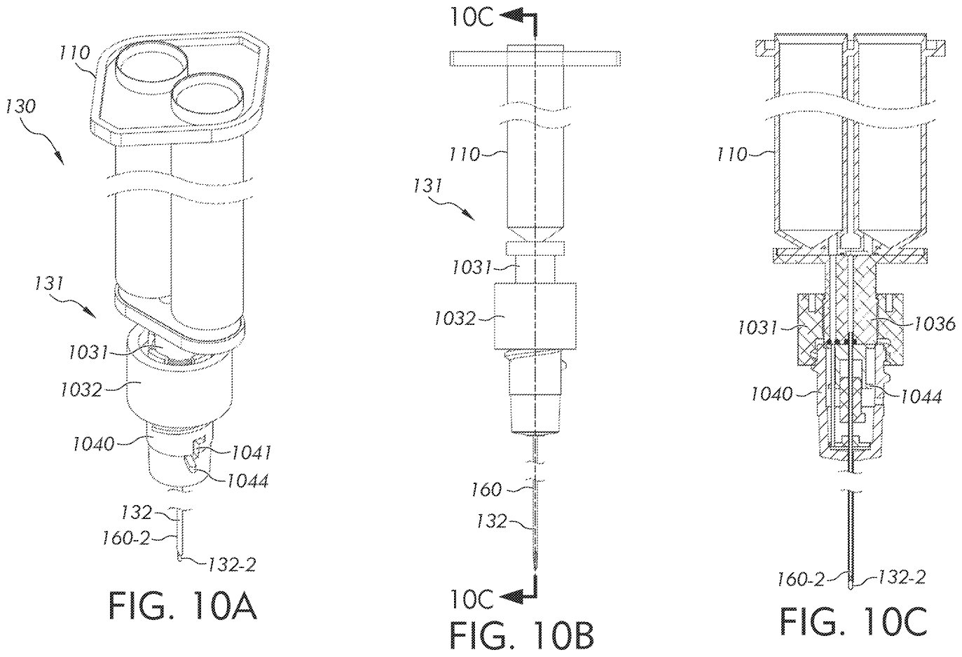

As further illustrated in FIG. 8, the elongate hollow stylet 132 may be disposed within the introducer cannula 160 such that the blunt, open distal end 132-2 of the elongate hollow stylet 132 is positioned proximally (e.g., in the −x-direction as depicted in the coordinate axis of FIG. 8) relative the beveled distal end 160-2 of the introducer cannula 160. In these embodiments, the elongate hollow stylet 132 may be translatable between an extended position (e.g., in which the blunt, open distal end 132-2 of the elongate hollow stylet 132 is positioned distally relative the beveled distal end 160-2 of the introducer cannula 160) and a retracted position (e.g., in which the blunt, open distal end 132-2 of the elongate hollow stylet 132 is positioned proximally relative the beveled distal end 160-2 of the introducer cannula 160), as will be described in additional detail herein with reference to FIGS. 10A-14C.

Referring still to FIG. 8, it should be further appreciated that, in the embodiments described herein, the distal end 130-2 of the injection needle assembly 130 may define a mixing chamber 151, which may be configured to allow the separate fluid components from the outer lumen 155 and the inner lumen 153 to mix before being deployed at a target area. For example, in these embodiments, the separate fluid components may enter the mixing chamber 151 separated, and may be combined (e.g., mixed) within the mixing chamber 151 before being deployed.

In these embodiments, translation of the elongate hollow stylet 132 may impact the mixing of the separate fluid components within the mixing chamber 151. For example, extending and/or retracting the elongate hollow stylet 132 may alter a volume of the mixing chamber 151, which may aid the mixing process by generating turbulence between the separate fluid components as the volume of the mixing chamber 151 changes. Furthermore, movement of the elongate hollow stylet 132 may further impact a flow direction of the separate fluid components, which may enhance mixing by generating a more dynamic fluid environment.

Although not depicted, in some embodiments, it should be further appreciated that the mixing chamber 151 may further include a valve, such as a one-way valve, which prevents the back flow of the fluid components into the inner and/or outer lumen 153, 155 once the separate fluid components have been mixed. In some embodiments, such a valve may be disposed proximally within the mixing chamber to prevent backflow of materials into the lumens 153, 155.

Furthermore, in some embodiments, the elongate hollow stylet 132 may include an inner hollow needle, such that the elongate hollow stylet 132 may define a first lumen (e.g., within the inner hollow needle) and a second lumen (e.g., between the inner hollow needle and the elongate hollow stylet 132). In these embodiments, the distal end 132-2 of the elongate hollow stylet 132 may be a sharpened and/or beveled distal end, while the inner hollow needle may include a blunted, open distal end.

In the embodiments described herein, the inner hollow needle may be extendable between an extended position and a retracted position, as has been described herein. For example, the inner hollow needle may be translatable between an extended position (e.g., in which the blunt, open distal end of the inner hollow needle is positioned distally relative the beveled distal end 132-2 of the elongate hollow stylet 132) and a retracted position (e.g., in which the blunt, open distal end of the inner hollow needle is positioned proximally relative the beveled distal end 132-2 of the elongate hollow style 132). Accordingly, in these embodiments, it should be appreciated that the injection needle assembly 130 may be capable of accessing a target area and supplying a material (e.g., hydrogel, etc.) to the target area without the introducer cannula 160.

Turning now to FIGS. 9A-9C, another embodiment of a delivery system 100 including the injection needle assembly 130 of FIG. 8 is depicted. In these embodiments, the delivery system 100 may include a material applicator 110 coupled to the injection needle assembly 130 via a hub 131, as has been described herein in detail with reference to FIGS. 1-4B and 8. Furthermore, the injection needle assembly 130 may include an elongate hollow stylet 132 disposed within an introducer cannula 160, as has been described herein in detail with reference to FIG. 8.

In these embodiments, the hub 131 of the injection needle assembly 130 may include an outer hub portion 1031 and an inner hub portion 1032, with the outer hub portion 1031 being rotatable relative the inner hub portion 1032. For example, in the embodiments depicted in FIGS. 9A-9C, the outer hub portion 1031 may be a threaded nut, while the inner hub portion 1032 may be a threaded rod, such that the outer hub portion 1031 may rotate about the inner hub portion 1032.

Referring still to FIGS. 9A-9C, in these embodiments, the introducer cannula 160 may further include a coaxial hub 162 which is configured to receive the elongate hollow stylet 132. In these embodiments, the coaxial hub 162 of the introducer cannula 160 may be further coupled to the inner hub portion 1032 of the hub 131 when the elongate hollow stylet 132 is positioned within the introducer cannula 160, such that the coaxial hub 162 is similarly capable of rotating relative to the inner hub portion 1032.

Furthermore, as most clearly depicted in FIG. 9C, in these embodiments, the proximal end 132-1 of the elongate hollow stylet 132 may further include a stylet hub 1036, which may be mechanically coupled to at least one of the coaxial hub 162 and/or the outer hub portion 1031 of the hub 131. Accordingly, in these embodiments, rotation of the coaxial hub 162 and/or the outer hub portion 1031 may cause the coaxial hub 162 and/or the outer hub portion 1031 may result in translation of the elongate hollow stylet 132 (e.g., in the +/−y-direction as depicted in the coordinate axes of FIGS. 9A-9C), as will be described in additional detail herein.

For example, as illustrated in FIGS. 9A-9C, and as described herein with reference to FIG. 8, the elongate hollow stylet 132 may be translatable between an extended position and a retracted position. In these embodiments, in a retracted position, the distal end 132-2 of the elongate hollow stylet 132 may be positioned distally relative the distal end 160-2 of the introducer cannula 160, such that the distal end 132-2 of the elongate hollow stylet 132 is enclosed within the introducer cannula 160. In these embodiments, with the distal end 160-2 of the introducer cannula 160 exposed, the beveled edge of the introducer cannula 160 may be used to pierce tissue at a target area (e.g., form an initial incision, etc.).

In these embodiments, rotation of the coaxial hub 162 and/or the outer hub portion 1031 of the hub 131 may be used to translate the elongate hollow stylet 132 from the retracted position to the extended position. For example, in embodiments in which the stylet hub 1036 is coupled to the outer hub portion 1031, rotation of the stylet hub 1036 in a first direction about the inner hub portion 1032 may cause the outer hub portion 1031 to move to an extended position (e.g., in the −y-direction as depicted in the coordinate axes of FIGS. 9A-9C). In these embodiments, the mechanical coupling of the stylet hub 1036 and the outer hub portion 1031 may similarly cause the stylet hub 1036, and in turn the elongate hollow stylet 132, move to the extended position, such that the distal end 132-2 of the elongate hollow stylet 132 extends from the distal end 160-2 of the introducer cannula 160. It should be appreciated that, in the embodiments described herein, the extension of the blunt, open distal end 132-2 of the elongate hollow stylet 132 from the introducer cannula 160 may effectively blunt the distal end 160-2 of the introducer cannula 160, such that the delivery system 100 may traverse the target area with minimal risk of the distal end 160-2 of the introducer cannula 160 perforating tissue at the target area.

Turning now to FIGS. 10A-10C, another embodiment of a delivery system 100 is depicted. In these embodiments, the hub 131 may further include the outer hub portion 1031 and the inner hub portion 1032, with the inner hub portion 1032 having a cam mechanism 1040. As provided herein, the term “cam mechanism” may refer to any mechanical component configured to convert rotational motion into linear motion, as will be described in additional detail herein.

Referring still to FIGS. 10A-10C, in these embodiments, the proximal end of the elongate hollow stylet 132 may further include a follower mechanism, such as a handle 1044, which is configured to engage the cam mechanism 1040. In the embodiments described herein, the handle 1044 may be integrally formed with the proximal end of the elongate hollow stylet 132, or may be fixedly joined with at least a portion of the proximal end 132-1 of the elongate hollow stylet 132, such that translation of the handle 1044 similarly results in translation of the elongate hollow stylet 132.

In these embodiments, the cam mechanism 1040 may further define a slot 1041 configured to receive the handle 1044. Accordingly, it should be appreciated that the slot 1041 may define the movement of the handle 1044 as the outer hub portion 1031 rotates, as will be described herein in detail with reference to FIGS. 12A-12G.

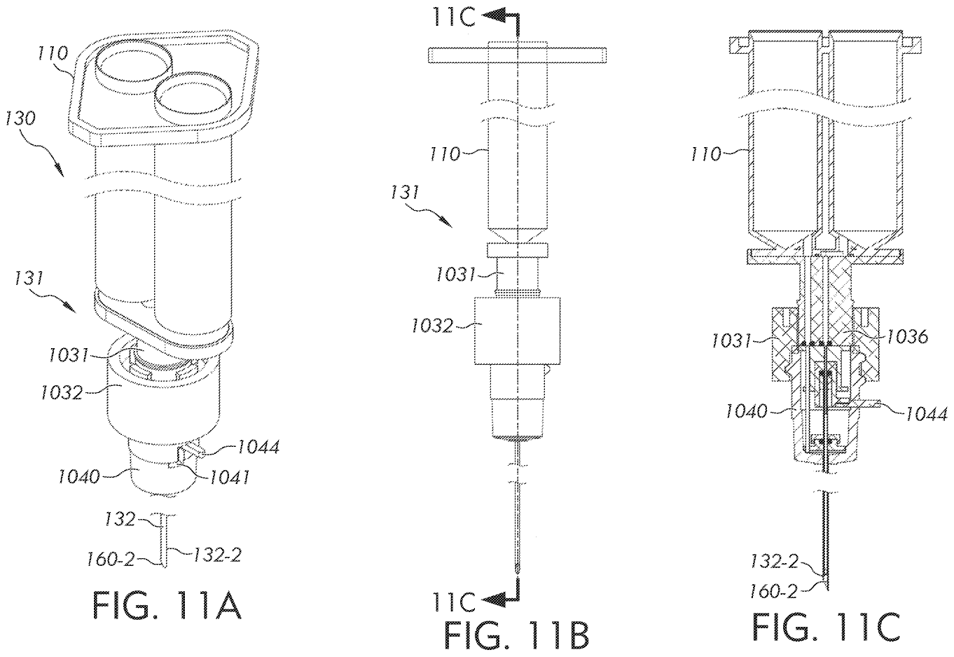

Referring still to FIGS. 10A-10C, it should be further appreciated that operation of the cam mechanism 1040 may act to translate the elongate hollow stylet 132 between the extended position and the retracted position. For example, FIGS. 10A-10C depict the elongate hollow stylet 132 in the extended position (e.g., with the distal end 132-2 of the elongate hollow stylet 132 extending distally beyond the distal end 160-2 of the introducer cannula 160), while FIGS. 11A-11C depict the same delivery system 100 with the elongate hollow stylet 132 in the retracted position (e.g., with the distal end 132-2 of the elongate hollow stylet 132 concealed within the distal end 160-2 of the introducer cannula 160).

Turning now to FIGS. 12A-12G, operation of the cam mechanism 1040 is depicted in additional detail. Initially, as depicted in FIG. 12A and described herein with reference to FIGS. 10A-11C, the outer hub portion 1031 may be disposed about the inner hub portion 1032 such that the outer hub portion 1031 is capable of rotating relative the inner hub portion 1032. For example, in these embodiments, the inner hub portion 1032 and the outer hub portion 1031 may each be threaded components, such that the outer hub portion 1031 is threadably engaged with the inner hub portion 1032.

In these embodiments, rotation of the outer hub portion 1031 relative the inner hub portion 1032 may cause the elongate hollow stylet 132 to translate between the extended position and the retracted position, as has been described herein. For example, as shown in FIG. 12B, rotating the outer hub portion 1031 in a first direction (e.g., counterclockwise) may cause the elongate hollow stylet 132 to translate from the extended position to the retracted position.

As depicted in FIGS. 12C and 12D, as the outer hub portion 1031 rotates in the first direction, the outer hub portion 1031 may engage the handle 1044 that is integrally formed with and/or otherwise affixed to the proximal end 132-1 of the elongate hollow stylet 132. In these embodiments, as the outer hub portion 1031 rotates, the outer hub portion 1031 may force the handle 1044 to traverse the slot 1041 of the cam mechanism 1040 in which the handle 1044 is situated.

For example, as illustrated in FIG. 12C, the first end 1041a of the slot 1041 may be positioned beneath (e.g., in the −y-direction as depicted in the coordinate axes of FIGS. 12A-12G) the second end 1041b of the slot 1041. Accordingly, as the handle 1044 translates between the first end 1041a of the slot 1041 and the second end 1041b of the slot, the handle 1044 may move upwardly (e.g., in the +y-direction as depicted in the coordinate axes of FIG. 12A-12G), which may result in the elongate hollow stylet 132 moving upwardly an equal distant. Accordingly, as the elongate hollow stylet 132 moves in the upward direction (e.g., in the +y-direction), the distal end 132-2 of the elongate hollow stylet 132 may be moved from the extended position to the retracted position, as described herein with reference to FIGS. 10A-11C.

Turning now to FIG. 12D, it should be appreciated that rotating the outer hub portion 1031 in a second direction opposite the first direction (e.g. clockwise) may allow the elongate hollow stylet 132 to move from the retracted position to the extended position. For example, as illustrated in FIGS. 12E and 12F, when the outer hub portion 1031 is rotated in the second direction, contact between the outer hub portion 1031 and the handle 1044 may cause the handle 1044 to move from the second end 1041b of the slot 1041 towards the first end 1041a of the slot 1041. In these embodiments, because the first end 1041a of the slot 1041 is positioned beneath the second end 1041b of the slot 1041, translating the handle 1044 from the second end 1041b of the slot 1041 towards the first end 1041a of the slot may cause the handle 1044, and in turn, the elongate hollow stylet 132 to move downwardly, such that the elongate hollow stylet 132 moves from the retracted position to the extended position.

In the embodiments described herein, it should be further appreciated that the configuration of the slot 1041 may be used to determine translation of the elongate hollow stylet 132. For example, reversing the orientation of the slot 1041 (e.g., such that the first end 1041a of the slot 1041 is positioned above the second end 1041b of the slot 1041) may similarly cause translation of the elongate hollow stylet 132 to be reversed, such that moving rotating the outer hub portion 1031 in the first direction extends the elongate hollow stylet 132 while rotating the outer hub portion 1031 in the second direction retracts the elongate hollow stylet 132.

Furthermore, it should be appreciated that adjusting a distance (e.g. in the +/−y-direction as depicted in the coordinate axes of FIGS. 12A-12G) between the first end 1041a and the second end 1041b of the slot 1041 may similarly adjust a distance in which the elongate hollow stylet 132 is translated. For example, in the embodiments described herein, the distance in which the elongate hollow stylet 132 moves between the retracted position and the extended position may be equal to the vertical distance between the first end 1041a of the slot 1041 and the second end 1041b of the slot 1041. Accordingly, increasing the vertical distance between the first end 1041a and the second end 1041b of the slot 1041 may increase the distance in which the elongate hollow stylet 132 translates, and vice versa.

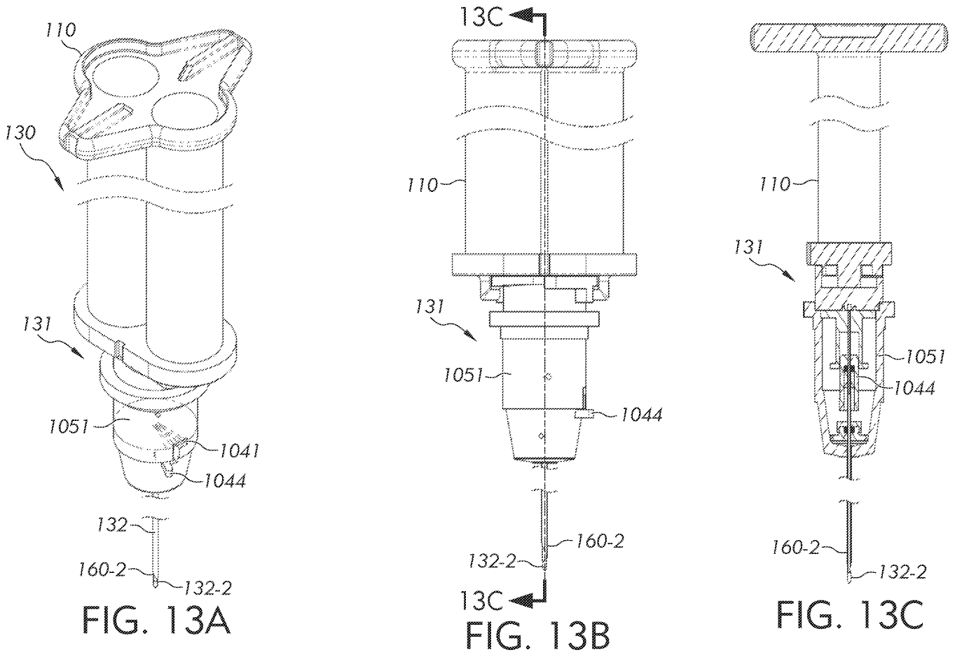

Referring still to FIGS. 12A-12G, the first end 1041a and the second end 1041b of the slot 1041 may act as locking mechanisms to secure the handle 1044 within the first end 1041a and the second end 1041b of the slot 1041, respectively, such that axial forces (e.g., in the +/−y-direction as depicted in the coordinate axes of FIGS. 13A-13G) do not act to translate the elongate hollow stylet 132 between the extended and retracted positions. For example, as depicted most clearly in FIGS. 12C and 12F, the first end 1041a of the slot 1041 and the second end 1041b of the slot 1041 may be sized and/or shaped to receive the handle 1044 when the elongate hollow stylet is translated to either the extended position or the retracted position. In these embodiments, the handle 1044 may be secured (e.g., locked, etc.) in either the first end 1041a or the second end 1041b of the slot 1041 such that the handle 1044 is only translatable within the slot 1041 when the outer hub portion 1031 is rotated to contact the handle 1044. By configured the slot 1041 as described herein, it may be possible to ensure that the elongate hollow stylet 132 is not inadvertently translated between the extended position and the retracted during operation of the delivery system 100.

Turning now to FIGS. 13A-13C, another embodiment of a delivery system 100 is depicted. In these embodiments, the hub 131 may include a hub body 1051 that further defines a slot 1041. Furthermore, the proximal end of the elongate hollow stylet 132 may further include a stylet hub 1036 that is rotatable within the hub body 1051.

As further illustrated in FIGS. 13A-13C, the stylet hub 1036 may include a follower mechanism, such as the handle 1044, which is configured to engage the slot 1041. In the embodiments described herein, the handle 1044 may be integrally formed with the proximal end of the elongate hollow stylet 132, or may be fixedly joined with at least a portion of the proximal end 132-1 of the elongate hollow stylet 132, such that translation of the handle 1044 similarly results in translation of the elongate hollow stylet 132.

Referring still to FIGS. 13A-13C, in these embodiments, the handle 1044 may be manually adjusted to move the handle 1044 through the slot 1041, and in turn, translate the elongate hollow stylet 132 between the extended position and the retracted position. For example, in these embodiments, a user may grasp the handle 1044 and move the handle 1044 from a first end 1041a of the slot 1041 to a second end 1041b of the slot 1041, as has been described in detail herein with reference to FIGS. 12A-12G. Accordingly, in the embodiments depicted in FIGS. 13A-13C, translation of the elongate hollow stylet 132 may be affected by manual translation of the handle 1044 as opposed to rotation of an element of the hub 131.

Referring now to FIGS. 9A-13C, it should be appreciated that, although the elongate hollow stylet 132 is depicted as including a blunt, open distal end 132-2 and the introducer cannula 160 is depicted as including a sharpened and/or beveled distal end 160-2, in some embodiments, the distal end 132-2 of the elongate hollow stylet 132 may be sharpened and/or beveled while the distal end 160-2 of the introducer cannula 160 may be blunted and/or opened. Accordingly, in these embodiments, the sharpened distal end 132-2 of the elongate hollow stylet 132 may be moved to the extended position to create an initial incision at a target area, and moved to the retracted position, to expose the blunt, open distal end 160-2 of the introducer cannula 160 to ensure that tissue at the target area is not inadvertently perforated during operation of the injection needle assembly 130.

It should now be understood that the present disclosure relates to multi-component delivery systems, such as systems for delivering a hydrogel (e.g., a hydrogel spacer) in various treatment procedures. The multi-component delivery systems described herein include a dual lumen syringe that delivers each component along separate paths such that the components mix at the point of delivery. In addition, the delivery systems described herein include auxiliary ports (e.g., a side port or the like) for delivery of flushing fluids or the like and also include extendable inner components that include a blunt tip and can be usable to avoid puncturing tissue when the needle is inserted in tissue. The delivery systems described herein include a dual chamber material applicator and an injection needle assembly, which are connectable to each other via one or more quick-connect/disconnect couplings. The delivery systems described herein are configured such that a user can quickly connect a dual syringe applicator to a needle assembly and deliver multi-component materials in a dual syringe system (e.g., components of a hydrogel) such that the components are mixed at the delivery site, the delivery needle can be flushed after delivery of the materials, and/or such that the delivery needle can be blunted after piercing tissue in order to avoid further puncturing tissue at a delivery site (e.g., prostate tissue and/or tissue surrounding a prostate).

Embodiments may be further described with references to the following numbered clauses:

Clause 1. An injection needle assembly, comprising: a hub comprising a first input port and a second input port, and an outer hub portion rotatably coupled about an inner hub portion having a cam; an elongate hollow stylet extending distally from the hub, the elongate hollow stylet having a proximal end at the hub and distal end spaced a distance from the proximal end; a cannula configured to receive the elongate hollow stylet such that the elongate hollow stylet and the cannula define an inner lumen and an outer lumen separate from the inner lumen, the cannula comprising a proximal end and a distal end spaced a distance from the proximal end; wherein the elongate hollow stylet is coupled to the hub such that the proximal end of the elongate hollow stylet engages with the cam and rotation of the outer hub portion translates the elongate hollow stylet between an extended position and a retracted position.

Clause 2. The injection needle assembly of clause 1, wherein the cam further includes a slot extending between a first end and a second end.

Clause 3. The injection needle assembly of clauses 1 or 2, wherein the proximal end of the elongate hollow stylet further includes a handle that engages the slot of the cam.

Clause 4. The injection needle assembly of any of clauses 1-3, wherein rotation of the outer hub portion in a first direction causes the outer hub portion to contact the handle and drive the handle between the first end of the slot and the second end of the slot.

Clause 5. The injection needle assembly of any of clauses 1-4, wherein rotation of the outer hub portion in a second direction opposite the first direction causes the outer hub portion to contact the handle and drive the handle between the second end of the slot and the first end of the slot.

Clause 6. The injection needle assembly of any of clauses 1-5, wherein the distal end of the elongate hollow stylet extends beyond the distal end of the cannula when the elongate hollow stylet is in the extended position.

Clause 7. The injection needle assembly of any of clauses 1-6, wherein the distal end of the elongate hollow stylet is contained within the distal end of the cannula when the elongate hollow stylet is in the retracted position.

Clause 8. The injection needle assembly of any of clauses 1-7, wherein the distal end of the elongate hollow stylet is an open, blunt distal end.

Clause 9. The injection needle assembly of any of clauses 1-8, wherein the distal end of the cannula is a sharpened, beveled distal end.

Clause 10. The injection needle assembly according to any of clauses 1-9, wherein the hub further comprises a connector shaped and sized to connect the hub to a material applicator.

Clause 11. The injection needle assembly according to any of clauses 1-10, wherein the connector is a quarter turn connector.

Clause 12. The injection needle assembly according to any of clauses 1-11, wherein the hub includes an auxiliary input port shaped and sized to be couplable to an auxiliary applicator.

Clause 13. The injection needle assembly of any of clauses 1-12, wherein the auxiliary input port is formed on a top surface of the hub and is positioned between the first input port and the second input port.

Clause 14. The injection needle assembly of any of clauses 1-13, wherein the auxiliary input port is formed within the material applicator.

Clause 15. A fluid delivery system, comprising: a dual chamber applicator comprising two chambers separate from one another, each chamber comprising at least one output port on a distal end thereof; and an injection needle assembly comprising: a hub comprising a first input port and a second input, and an outer hub portion rotatably coupled about an inner hub portion having a cam; an elongate hollow stylet extending distally from the hub, the elongate hollow stylet having a proximal end at the hub and distal end spaced a distance from the proximal end; a cannula configured to receive the elongate hollow stylet such that the elongate hollow stylet and the cannula define an inner lumen and an outer lumen separate from the inner lumen, the cannula comprising a proximal end and a distal end spaced a distance from the proximal end; wherein the elongate hollow stylet is coupled to the hub such that the proximal end of the elongate hollow stylet engages with the cam and rotation of the outer hub portion translates the elongate hollow stylet between an extended position and a retracted position.

Clause 16. The fluid delivery system of clause 15, wherein the cam further includes a slot extending between a first end and a second end.

Clause 17. The fluid delivery system of clause 15 or 16, wherein the proximal end of the elongate hollow stylet further includes a handle that engages the slot of the cam.

Clause 18. The fluid delivery system of any of clauses 15-17, wherein rotation of the outer hub portion in a first direction causes the outer hub portion to contact the handle and drive the handle between the first end of the slot and the second end of the slot.

Clause 19. The fluid delivery system of any of clauses 15-18, wherein rotation of the outer hub portion in a second direction opposite the first direction causes the outer hub portion to contact the handle and drive the handle between the second end of the slot and the first end of the slot.

Clause 20. The fluid delivery system of any of clauses 15-19, wherein the distal end of the elongate hollow stylet extends beyond the distal end of the cannula when the elongate hollow stylet is in the extended position.

Clause 21. The fluid delivery system of any of clauses 15-20, wherein the distal end of the elongate hollow stylet is contained within the distal end of the cannula when the elongate hollow stylet is in the retracted position.

Clause 22. The fluid delivery system of any of clauses 15-21, wherein the distal end of the elongate hollow stylet is an open, blunt distal end.

Clause 23. The fluid delivery system of any of clauses 15-22, wherein the distal end of the cannula is a sharpened, beveled distal end.

Clause 24. A method of translating an elongate hollow stylet in an injection needle assembly, the method comprising: forming a handle on a proximal end of the elongate hollow stylet; disposing the elongate hollow stylet within a cannula, such that a distal end of the elongate hollow stylet extends beyond a distal end of the cannula; engaging the handle with a slot formed in a cam mechanism of an inner hub portion of a hub of the injection needle assembly, the hub further including an outer hub portion rotatably coupled to the inner hub portion; and rotating the outer hub portion relative the inner hub portion, such that the outer hub portion contacts the handle and drives the handle from a first end of the slot to a second end of the slot; wherein translation of the handle from the first end of the slot to the second end of the slot causes the elongate hollow stylet to retract, such that the distal end of the elongate hollow stylet is enclosed within the cannula.

The terminology used herein is for the purpose of describing particular aspects only and is not intended to be limiting. As used herein, the singular forms “a,” “an,” and “the” are intended to include the plural forms, including “at least one,” unless the content clearly indicates otherwise. “Or” means “and/or.” As used herein, the term “and/or” includes any and all combinations of one or more of the associated listed items. It will be further understood that the terms “comprises” and/or “comprising,” or “includes” and/or “including” when used in this specification, specify the presence of stated features, regions, integers, steps, operations, elements, and/or components, but do not preclude the presence or addition of one or more other features, regions, integers, steps, operations, elements, components, and/or groups thereof. The term “or a combination thereof” means a combination including at least one of the foregoing elements.

It is noted that the terms “substantially” and “about” may be utilized herein to represent the inherent degree of uncertainty that may be attributed to any quantitative comparison, value, measurement, or other representation. These terms are also utilized herein to represent the degree by which a quantitative representation may vary from a stated reference without resulting in a change in the basic function of the subject matter at issue.

While particular embodiments have been illustrated and described herein, it should be understood that various other changes and modifications may be made without departing from the spirit and scope of the claimed subject matter. Moreover, although various aspects of the claimed subject matter have been described herein, such aspects need not be utilized in combination. It is therefore intended that the appended claims cover all such changes and modifications that are within the scope of the claimed subject matter.

1. An injection needle assembly, comprising:
a hub comprising a first input port and a second input port, and an outer hub portion rotatably coupled about an inner hub portion having a cam;
an elongate hollow stylet extending distally from the hub, the elongate hollow stylet having a proximal end at the hub and a distal end spaced a distance from the proximal end;
a cannula configured to receive the elongate hollow stylet such that the elongate hollow stylet and the cannula define an inner lumen and an outer lumen separate from the inner lumen, the cannula comprising a proximal end and a distal end spaced a distance from the proximal end;
wherein the elongate hollow stylet is coupled to the hub such that the proximal end of the elongate hollow stylet engages with the cam and rotation of the outer hub portion translates the elongate hollow stylet between an extended position and a retracted position.

2. The injection needle assembly of claim 1, wherein the cam further includes a slot extending between a first end and a second end.

3. The injection needle assembly of claim 2, wherein the proximal end of the elongate hollow stylet further includes a handle that engages the slot of the cam.

4. The injection needle assembly of claim 3, wherein rotation of the outer hub portion in a first direction causes the outer hub portion to contact the handle and drive the handle between the first end of the slot and the second end of the slot.

5. The injection needle assembly of claim 4, wherein rotation of the outer hub portion in a second direction opposite the first direction causes the outer hub portion to contact the handle and drive the handle between the second end of the slot and the first end of the slot.

6. The injection needle assembly of claim 1, wherein the distal end of the elongate hollow stylet extends beyond the distal end of the cannula when the elongate hollow stylet is in the extended position.

7. The injection needle assembly of claim 1, wherein the distal end of the elongate hollow stylet is contained within the distal end of the cannula when the elongate hollow stylet is in the retracted position.

8. The injection needle assembly of claim 1, wherein the distal end of the elongate hollow stylet is an open, blunt distal end.

9. The injection needle assembly of claim 1, wherein the distal end of the cannula is a sharpened, beveled distal end.

10. The injection needle assembly of claim 1, wherein the hub further comprises a connector shaped and sized to connect the hub to a material applicator.

11. The injection needle assembly according to claim 10, wherein the connector is a quarter turn connector.

12. The injection needle assembly according to claim 1, wherein the hub includes an auxiliary input port shaped and sized to be couplable to an auxiliary applicator.

13. The injection needle assembly according to claim 1, wherein the auxiliary input port is formed on a top surface of the hub and is positioned between the first input port and the second input port.

14. The injection needle assembly of claim 13, wherein the auxiliary input port is formed within the material applicator.

15. A fluid delivery system, comprising:
a dual chamber applicator comprising two chambers separate from one another, each chamber comprising at least one output port on a distal end thereof; and
the injection needle assembly according to claim 1.

16. (canceled)

17. (canceled)

18. (canceled)

19. (canceled)

20. (canceled)

21. (canceled)

22. (canceled)

23. (canceled)

24. A method of translating an elongate hollow stylet in an injection needle assembly, the method comprising:
forming a handle on a proximal end of the elongate hollow stylet;
disposing the elongate hollow stylet within a cannula, such that a distal end of the elongate hollow stylet extends beyond a distal end of the cannula;
engaging the handle with a slot formed in a cam mechanism of an inner hub portion of a hub of the injection needle assembly, the hub further including an outer hub portion rotatably coupled to the inner hub portion; and
rotating the outer hub portion relative the inner hub portion, such that the outer hub portion contacts the handle and drives the handle from a first end of the slot to a second end of the slot;
wherein translation of the handle from the first end of the slot to the second end of the slot causes the elongate hollow stylet to retract, such that the distal end of the elongate hollow stylet is enclosed within the cannula.
